(19) United States
(12) Patent Application Publication
(10) Pub. No.: US 2002/0137696 A1
(43) Pub. Date: Sep. 26, 2002

(54) Specific modulation of TH1/TH2 cytokine expression by ribavirin in activated T-lymphocytes

(76) Inventor: Robert Tam, Irvine, CA (US)

Correspondence Address: Robert D. Fish, Suite 706, 1440 N. Harbor Blvd., Fullerton, CA, 92835, US

(21) Appl. No.: 10/094,032

(22) Filed: Mar. 8, 2002

Related U.S. Application Data
(62) Division of application No. 09/624,855, filed on Jul. 25, 2000.
Application No. 09/624,855 is a continuation-in-part of application No. 09/156,646, filed on Sep. 18, 1998, now Pat. No. 6,150,337.
Application No. 09/156,646 is a continuation-in-part of application No. 09/097,450, filed on Jun. 15, 1998, now Pat. No. 6,063,772.
Application No. 09/097,450 is a continuation of application No. 08/590,449, filed on Jan. 23, 1996, now Pat. No. 5,767,097.

(51) Int. Cl. A61K 31/7056

(52) U.S. Cl. 514/43

(57) ABSTRACT

Ribavirin is employed in a manner which is effective to modulate lymphokine expression in activated T cells. In particular, Ribavirin is used to suppress Type 2-mediated T cell responses and promote Type 1-mediated T cell response. Thus, instead of administering Ribavirin in its well-recognized role as an anti-viral agent, Ribavirin is herein used in the treatment of imbalances in lymphokine expression. Such imbalances may be found to be concomitants of allergic atopic disorders such as allergic asthma and atopic dermatitis, helminth infection and leishmaniasis, and various primary and secondary immunodeficiencies, which may or may not also be associated with viral infection.

This application is a continuation-in-part of, allowed U.S. Ser. No. 09/156,646, filed Sep. 18, 1998, which is a continuation in part of U.S. Pat. No. 09/097450, filed Jun. 15, 1998, issued on May 16, 2000 as U.S. Pat. No. 6,063,772, which is a continuation of U.S. Ser. No. 08/590449 filed Jan. 23, 1996, issued on Jun. 16, 1998 as U.S. Pat. No. 5,767,097.

### FIELD OF THE INVENTION

The field of the invention is immunology.

### BACKGROUND OF THE INVENTION

From seminal work by Mossman and Coffman (Mossmann T R, Coffins R L: Th1 and Th2 cells: different patterns of lymphokine secretion lead to different functional properties. Annu Rev Immunol 1989, 7: 145-173), growth factors known as cytokines produced by T helper or CD4⁺ T cells in both human and murine systems were classified into two subsets, Th1 and Th2. These were characterized by their functions in regulating various types of immune responses. Cytokines produced by Th1 cells [interleukin (IL)-2, interferon-alpha (IFNγ), tumor necrosis factor-alpha (TNFα), IL-12] stimulated strong cellular immunity whereas Th2 cytokines [IL-4, IL-5, IL-6, IL-10, IL-13] were important for eliciting humoral (antibody) responses in vivo. Recently cytokines produced by non-CD4⁺ T cells have been shown to be important in in vivo responses. In particular, the cytotoxic or CD8⁺ T cells can also be subdivided into two subgroups, Tc1 and Tc2, which correspond to the same subsets in T helper cells (Carter LL, Dutton RW: Type 1 and Type 2: a functional dichotomy for all T cell subsets. Curr Opin Immunol 1996, 8: 336-342). This has led to the current nomenclature being generalized from Th1/Th2 to Type1/Type 2 to reflect more closely the response generated by particular cytokines, rather than the cell types that produces them.

At the time the original application was filed for the recently issued patent (Specific modulation of Th1/Th2 cytokine expression by ribavirin in activated T cells—R. Tam, U.S. Pat. No. 5,767,097), the nomenclature of Type 1 and Type 2 had not been universally adopted. We thus used the Th1/Th2 nomenclature prevalent at the time of the original filing to include both CD4⁺and CD8⁺ T cells, as shown in the ‘Background’ section of that application (column 1, line 14). In this application we employ the terms, Type 1 and Type 2, instead of the previously used terms, Th1/Th2.

Strongly polarized Type 1 and Type 2 responses not only play different roles in protection, they can promote different immunopathological reactions. Type 1-type responses are involved organ specific autoimmunity such as experimental autoimmune uveoretinitis (Dubey et al, 1991, Eur Cytokine Network 2: 147-152), experimental autoimmune encephalitis (EAE) (Beraud et al, 1991, Cell Immunol 133 : 379-389) and insulin dependent diabetes mellitus (Hahn et al,1987, Eur J Immunol 18 : 2037-2042), in contact dermatitis (Kapsenberg et al, Immunol Today 12: 392-395), and in some chronic inflammatory disorders. In contrast Type 2-type responses are responsible for triggering allergic atopic disorders (against common environmental allergens) such as allergic asthma (Walker et al, 1992, Am Rev Resp Dis 148: 109-115) and atopic dermatitis (van der Heijden et al, 1991, J Invest Derm 97 : 389-394), are thought to exacerbate infection with tissue-dwelling protozoa such as helminths (Finkelman et al, 1991, Immunoparasitol Today 12: A62-66) and Leishmania major (Caceres-Dittmar et al, 1993, Clin Exp Immunol 91: 500-505), are preferentially induced in certain primary immunodeficiencies such as hyper-IgE syndrome (Del Prete et al, 1989, J Clin Invest 84: 1830-1835) and Omenn's syndrome (Schandene et al, 1993, Eur J Immunol 23: 56-60), and are associated with reduced ability to suppress HIV replication (Barker et al, 1995, Proc Soc Nat Acad Sci U.S.A 92: 11135-11139).

Thus, it is clear that modulation of the lymphokine profiles of the aforementioned disease states would be of therapeutic benefit. Promoting a Type 1 response would most likely lead to the reversal of a Type 2 phenotype and vice versa. Monoclonal antibodies (mAb) to lymphokines, lymphokines themselves and other agents such as thiol antioxidants (Jeannin et al, 1995, J Exp Med 182: 1785-1792) have been shown to reverse the pathogenesis of certain diseases by inhibiting the disease-promoting cytokine pattern, either Type 1 or Type 2. For example, intracellular protozoan infections are limited by IFNγ but exacerbated by IL-4, while nematode infections are controlled by IL-4 and exacerbated by IFNα (Heinzel et al, 1989, J Exp Med 162: 59-72, Else et al, 1994, J Exp Med 179: 347-351). Insulin-dependent diabetes mellitus in NOD mice and EAE in mice and rats can be ameliorated by treatment with IL-4 or anti- IFNγ mAb before development of the disease (Rapoport et al, 1993, J Exp Med 178: 87-99, Racke et al, 1994, J Exp Med 180: 1961-1966, Campbell et al, 1991, J Clin Invest 87: 739-742). In addition, autoimnmune graft versus host disease (GVHD) that is characterized by a systemic lupus erythrematosus-like syndrome is associated with Type 2 lymphokine production and is inhibited by anti-IL-4 antibody (Umland et al, 1992, Clin Immunol Immunopathol 63: 66-73). On the other hand, Type 1 cytokines are produced in acute GVHD, in which donor CD8⁺ T cells develop into CTL and destroy the host immune system. Treatment with anti-IFNγ or anti-TNFα mAb ameliorates disease, and treatment with anti-IL-2 mAb converts acute GVHD to autoimmune GVHD (Via and Finkelman, 1993, Int Immunol 5: 565-572).

Clinical trials of native and recombinant IL-2 in treating HIV-infected patients have been in progress since 1983 (Volberding et al, 1987, AIDS Res Hum Retroviruses, 3: 115-124). Here, the relationship comes from the fact that development of AIDS has been reported to be associated with a shift in the pattern of lymphokines produced (Clerici and Shearer, 1994, Immunol Today 15: 575-581). Over time, in an infected individual progressing towards disease, a decreased expression of Type 1 lymphokines such as IL-2 occurs (Maggi et al, 1987, Eur J Immunol 17: 1685-1690, Gruters et al, 1990, Eur J Immunol 20: 1039-1044, Clerici et al, 1993, J Clin Invest 91: 759-765), concomitant with an increased production of Type 2 lymphokines such as IL-4 and IL-10 (Clerici et al, 1994, J Clin Invest 93: 768-775, Hoffman et al, 1985, Virology 147: 326-335). T-cells from asymptomatic or long term survivors treated with IL-2 enhanced their anti-HIV activity whereas exposure to IL-4 or IL-10 reduced their ability to suppress HIV replication and to produce IL-2 (Barker et al, 1995, Proc Soc Nat Acad Sci U.S.A 92: 11135-11139).

These current immunomodulatory therapeutics (mAbs and recombinant cytokines) are, however, not without limitations. For example with chronic monoclonal antibody treatment, the host animal develops antibodies against the monoclonal antibodies thereby limiting their usefulness. ‘Humanized’ monoclonal antibodies have been developed which apparently reduces the risk of an induced immune response to these mAbs. However, these are still under development, and in addition these new mAbs remain large proteins and therefore may have difficulty reaching there target sites. Cytokine-based therapeutics also have limitations. For example, IL- 12 treatment of autoimmune GVHD leads to the development of acute GVHD in mice.

Ribavirin (1-β-D-ribofuranosyl- 1,2,4-triazole-3 -carboxamide) is a synthetic nucleoside capable of inhibiting RNA and DNA virus replication (Huffman et al, 1973, Antimicrob. Agents Chemother 3: 235, Sidwell et al, 1972, Science 177: 705). We have confirmed the observations of others who suggested that Ribavirin, in addition to its antiviral activity, has an effect on certain immune responses (reviewed Jolley and Suchil, 1984, Clinical Applications of Ribavirin: p93-96). We have also confirmed observations of others that Ribavirin affects the proliferation of mitogen- and antigen-activated T and B lymphocytes, (Tam et al, 1995 (data not shown), Peavy et al, 1980, Infection and Immunity 29: 583-589) and then when combined with cyclosporin, Ribavirin showed efficacy in long term allograft survival, Jolley et al (1988, Transplantation Proc 20: 703-706).

In addition, we have significantly advanced the prior research by demonstrating that Ribavirin modulates the cytokine pattern of an immune response at least in part by promoting a Type 1 response and suppressing a Type 2 response. In hindsight, this discovery is not inconsistent with prior research. First, Ribavirin is known to inhibit both functional humoral immune responses, (Peavy et al, 1981, J Immunol 126: 861-864, Powers et al, 1982, Antimicrob Agents Chemother 22: 108-114) and IgE-mediated modulation of mast cell secretion (Marquardt et al, 1987, J Pharmacol Exp Therapeutics 240: 145-149, (both Type 2 lymphokine-mediated events). Second, Ribavirin antagonizes the antiviral effect of azidothymidine (AZT) in peripheral blood lymphocytes from HIV patients (Vogt et al, 1987, Science 235: 1376-1379). This finding is significant because AZT decreases IL-2 receptor (IL-2R) but not IL-2 expression (Viora and Camponeschi, 1995, Cell Immunol 163: 289-295). It is therefore possible that Ribavirin antagonizes AZT by modulating IL-2 expression and elevating depressed levels of IL-2R. Third, Ribavirin treatment of an immunocompromised patient for chronic GVHD (a Type 2-mediated disorder) led to a dramatic resolution of the disease, an outcome which did not occur with conventional immunosuppressive therapies such as cyclosporin and glucocorticoids (Cassano, 1991, Bone Marrow Transplantation 7: 247-248). Finally, Ribavirin treatment (one year) of patients for hepatitis C (HCV) revealed fewer lymphocyte aggregates and far less liver damage than placebo controls (Dusheiko et al, 1994, Hepatology 20: 206A). This observation may reflect the fact that although, the predominant immune response to hepatitis C is mediated by Type 1 lymphokines, T cells of the Type 2 phenotype can be infected by HCV (Zignego et al, 1994, unpublished data) and this infection may drive further antibody-mediated destruction of hepatocytes.

### BRIEF DESCRIPTION OF THE DRAWINGS

FIG. 1A is a graphical representation of the effect of Ribavirin and interferon alpha on the extracellular expression of IL-2 in PMA/ionomycin-activated T lymphocytes. Results are expressed as percentage of the increased lymphokine expression following PMA/ionomycin treatment alone.

FIG. 1B is a graphical representation of the effect of Ribavirin and interferon alpha on the extracellular expression of TNFA in PMA/ionomycin-activated T lymphocytes. Results are expressed as percentage of the increased lymphokine expression following PMA/ionomycin treatment alone.

FIG. 1C is a graphical representation of the effect of Ribavirin and interferon alpha on the extracellular expression of IL-4 in PMA/ionomycin-activated T lymphocytes. Results are expressed as percentage of the increased lymphokine expression following PMA/ionomycin treatment alone.

FIG. 1D is a graphical representation of the effect of Ribavirin and interferon alpha on the extracellular expression of IFNγ in PMA/ionomycin-activated T lymphocytes. Results are expressed as percentage of the increased lymphokine expression following PMA/ionomycin treatment alone.

FIG. 2A is a graphical representation of the effect of 2, 10 or 50 μM Ribavirin in the presence of 2000 U/ml interferon alpha (left panels) and the effect of 500, 1000 or 2000 U/ml interferon alpha (right panels)in the presence of 10 μM Ribavirin on the extracellular expression of IL-2 in PMA/ionomycin-activated T lymphocytes.

FIG. 2B is a graphical representation of the effect of 2, 10 or 50 μM Ribavirin in the presence of 2000 U/ml interferon alpha (left panels) and the effect of 500, 1000 or 2000 U/ml interferon alpha (right panels)in the presence of 10 μM Ribavirin on the extracellular expression of IL-4 in PMA/ionomycin-activated T lymphocytes.

FIG. 2C is a graphical representation of the effect of 2, 10 or 50 μM Ribavirin in the presence of 2000 U/ml interferon alpha (left panels) and the effect of 500, 1000 or 2000 U/ml interferon alpha (right panels)in the presence of 10 μM Ribavirin on the extracellular expression of IL-2 in PMA/ionomycin-activated T lymphocytes.

FIG. 2D is a graphical representation of the effect of 2, 10 or 50 μM Ribavirin in the presence of 2000 U/ml interferon alpha (left panels) and the effect of 500, 1000 or 2000 U/ml interferon alpha (right panels)in the presence of 10 μM Ribavirin on the extracellular expression of IL-4 in PMA/ionomycin-activated T lymphocytes.

FIG. 3 is a graphical representation of the effect of Ribavirin and interferon alpha on IL-2, IL-4 and IFNγ mRNA expression in PMA/ionomycin-activated T lymphocytes.

FIG. 4A is a graphical representation of the effect of Ribavirin and interferon alpha on the cell surface expression of IL-2 receptors in PMA/ionomycin-activated T lymphocytes. Results are expressed as percentage of the increased lymphokine receptor expression following PMA/ionomycin treatment alone.

FIG. 4B is a graphical representation of the effect of Ribavirin and interferon alpha on the cell surface expression of IL-4 receptors in PMA/ionomycin-activated T lymphocytes. Results are expressed as percentage of the increased lymphokine receptor expression following PMA/ionomycin treatment alone.

FIG. 5A is a graphical representation of the expression of intracellular IL-2 expression in resting CD4⁺ T cells. Data from one experiment is shown and represented as the percentage of cells showing double positive staining for IL-2 and CD4 or CD8.

FIG. 5B is a graphical representation of the expression of intracellular IL-2 expression in activated CD4⁺ T cells treated with PMA/ionomycin alone. Data from one experiment is shown and represented as the percentage of cells showing double positive staining for IL-2 and CD4 or CD8.

FIG. 5C is a graphical representation of the expression of intracellular IL-2 expression in activated CD4⁺ T cells in the presence of 10 μM Ribavirin. Data from one experiment is shown and represented as the percentage of cells showing double positive staining for IL-2 and CD4 or CD8.

FIG. 5D is a graphical representation of the expression of intracellular IL-2 expression in activated CD4⁺ T cells treated with 5000 U/ml interferon alpha. Data from one experiment is shown and represented as the percentage of cells showing double positive staining for IL-2 and CD4 or CD8.

FIG. 5E is a graphical representation of the expression of intracellular IL-2 expression in resting CD8⁺ T cells. Data from one experiment is shown and represented as the percentage of cells showing double positive staining for IL-2 and CD4 or CD8.

FIG. 5F is a graphical representation of the expression of intracellular IL-2 expression in activated CD8⁺ T cells treated with PMA/ionomycin alone. Data from one experiment is shown and represented as the percentage of cells showing double positive staining for IL-2 and CD4 or CD8.

FIG. 5G is a graphical representation of the expression of intracellular IL-2 expression in activated CD8⁺ T cells in the presence of 10 μM Ribavirin. Data from one experiment is shown and represented as the percentage of cells showing double positive staining for IL-2 and CD4 or CD8.

FIG. 5H is a graphical representation of the expression of intracellular IL-2 expression in activated CD8⁺ T cells treated with 5000 U/ml interferon alpha. Data from one experiment is shown and represented as the percentage of cells showing double positive staining for IL-2 and CD4 or CD8.

FIG. 6A is a graphical representation of a contemplated Ribavirin analog.

FIG. 6B is a graphical representation of a contemplated Ribavirin analog.

FIG. 6C is a graphical representation of a contemplated Ribavirin analog.

FIG. 6D is a graphical representation of a contemplated Ribavirin analog.

FIG. 7A is a graph showing the results of various concentrations of Ribavirin analogs on IL-2.

FIG. 7B is a graph showing the results of various concentrations of Ribavirin analogs on TNF-α.

FIG. 7C is a graph showing the results of various concentrations of Ribavirin analogs by on IFN-γ.

FIG. 7D is a graph showing the results of various concentrations of Ribavirin analogs on IL-4.

FIG. 7E is a graph showing the results of various concentrations of Ribavirin analogs on IL-5.

### SUMMARY OF THE INVENTION

In accordance with the present invention, the nucleoside, Ribavirin, is administered to a patient in a dosage range which is effective to modulate lymphokine expression in activated T cells. In particular, Ribavirin is used to suppress Type 2-mediated T cell responses and promote Type 1-mediated T cell response.

Thus, instead of administering Ribavirin in its well-recognized role as an anti-viral agent, Ribavirin is herein used in the treatment of imbalances in lymphokine expression. Such imbalances may be found to be concomitants of allergic atopic disorders such as allergic asthma and atopic dermatitis, helminth infection and leishmaniasis, and various primary and secondary immunodeficiencies, which may or may not also be associated with viral infection.

### DETAILED DESCRIPTION OF SPECIFIC EMBODIMENTS

Ribavirin is preferably administered orally to a human patient in a dosage which achieves a blood serum level averaging about 0.25 to about 6.7 μM.”

TABLE 1

| Mg/day | mg/kg/day | μM |
|---|---|---|
| 800 | 11.4 | 6.7 |
| 600 | 8.6 | 5.0 |
| 400 | 5.7 | 3.3 |
| 300 | 4.3 | 2.5 |
| 200 | 2.9 | 1.7 |
| 125 | 1.8 | 1.0 |
| 60 | 0.9 | 0.50 |
| 30 | 0.4 | 0.25 |

Table 2, for comparison, gives the previously known dosage ranges.

TABLE 2

| mg/day | mg/kg/day | μM | |
|---|---|---|---|
| 1500 | 21.4 | 12.5 | highest level of oral administration |
| 1200 | 17.1 | 10.01 | level at which anemia is problematic |
| 1000 | 14.3 | 8.31 | lowest level of prior art antiviral use |

Since Ribavirin has been on the market for several years, many dosage forms and routes of administration are known, and all appropriate dosage forms and routes of administration may be utilized. For example, in addition to oral administration, Ribavirin may given intravenously, intramuscularly, intraperitoneally, topically, and the like, all of which are known. Pharmaceutical formulations comprising Ribavirin may also comprise one or more pharmaceutically acceptable carriers, which may include excipients such as stabilizers (to promote long term storage), emulsifiers, binding agents, thickening agents, salts, preservatives, solvents, dispersion media, coatings, antibaterial and antifungal agents, isotonic and absorption delaying agents and the like. The use of such media and agents for pharmaceutical active substances is well known in the art. Except insofar as any conventional media or agent is incompatible with the Ribavirin, its use in labeled monoclonal antibodies were obtained from Becton Dickinson (San Jose, Calif.) except for anti-CDw124 which was obtained from Pharmingen, San Diego, Calif. Incubations were performed at 4° C. in the dark for 45 min using saturating mAb concentrations. Unincorporated label was removed by washing in PBS prior to the analysis with a FACScan flow cytometer (Becton Dickinson).

Antigen density was indirectly determined in gated live CD4⁺ T cells and expressed as the mean channel of fluorescence (MCF). Surface expression of specific antigen (CDw124, CD25) was represented as the mean channel shift (MCS) obtained by subtracting the MCF of FITC- or PE-labeled isotype-matched (IgG1) control mAb-stained cells from the MCF of FITC- or PE-labeled antigen-specific mAb stained cells. Alternatively, surface expression of the CD4⁺ -subset of cells stained with CD28 mAb was determined by subtracting the MCF of CD28⁺ CD4⁺ from the MCF of CD28⁻ CD4⁻ cells.

The viability of control untreated and Ribavirin and interferon α-treated cells were determined in each batch of all oligonucleotides in multiple donors by staining with the vital dye, propidium iodide (5 μg/ml final concentration). The percentage of live cells which excluded propidium iodide was determined by flow cytometry and was >90% (range 90-99%) following treatment with all concentrations used.

Immunofluorescence Analyses of Intracellular Cytokine Expression

For analyses of the intracellular expression of IL-2 in CD4⁺ and CD8⁺ T cell subsets, T cells were first treated for the last 4 h of 48-72 h activation with 10 μg Brefeldin A (Gibco BRL, Gaithersburg, Md.) to minimize secretion of newly synthesized IL-2 into the extracellular milieu. Following activation, 900 μl cell supernatant from each microplate was transferred to another microplate for analysis of cell-derived cytokine production. Prior to direct staining (30 min, 4 C., in the dark) with FITC-conjugated antibodies to the cell surface antigens, CD4 and CD8, the cells were washed twice with isotonic saline solution, pH 7.4 and resuspended in 100-150 μl Staining Buffer (phosphate buffered saline, pH 7.4 containing 1% Fetal Calf Serum (FCS) (Hyclone, Logan, Utah) and 0.1% sodium azide), and split into two samples. Stained cells were washed in 1 ml Staining Buffer and cell pellet resuspended in 100 μl Fixation Buffer (4% paraformaldehyde in PBS) following aspiration of the supernatant. Fixed cells were kept at 4 C. for 20 mins, then washed in 1 ml Staining Buffer and cell pellet resuspended with mixing in 50 μl Perneabilization Buffer (0.1% saponin (ICN, Costa Mesa, Calif.) in PBS). Permeabilized cells were stained with PE-labeled IL-2 antibody for 30 mm at 4 C. in the dark and then washed in 1 ml Permeabilization Buffer, resupended in 250 μl Staining Buffer prior to FACS analysis.

Analysis of Cytokine mRNA

Total RNA was extracted from resting T cells and from Ribavirin and interferon α-treated and untreated activated T cells using a commercial variation of the guanidium thiocyanate/phenol extraction technique (Trizol reagent (GIBCO/BRL). RNA was washed with 70% ethanol and finally resuspended in 10 μl DEPC-treated water.

cDNA synthesis reaction was performed as per manufacturers instructions (Promega, Madion, Wis.). Briefly, 1 μg of total RNA was heated at 65° C. for 10 min and cooled on ice before combining with 2 μl 10× reverse transcription buffer (100 mM Tris HCl (pH 8.8), 500 mM KCl, 1% Triton X-100), 5 mM MgCl, 2 μl 10 mM dNTPs (1 mM each dNTP), 0.5 μl RNase inhibitor, 1 μl oligo (dT)₁₅ primer (0.5 μg/μg RNA) and 0.65 μl AMV reverse transcriptase (H.C.). The reaction was incubated at 42° C. for 1 h followed by at 95° C. for 10 min and 5 min on ice.

The PCR reaction was performed using GeneAmp PCR kit (Perkin-Elmer Cetus, Foster City, Calif.). In a fresh tube, RT reaction mixture (3 μl) was combined with 5 μl10× PCR buffer (500 mM KCl, 100 mM Tris-HCl, pH 8.3, 15 mM MgCl₂ and 0.01% (w/v) gelatin), 1 μl 10 mM dNTPs and 1 U of Taq DNA polymerase. The primers used were as follows: interleukin-2, interleukin-4, interferon-γ (human) primers (Stratagene, La Jolla, Calif.) and pHE7 ribosomal gene. Amplification conditions were 45 sec at 94° C., 1 min at 57° C. and 2 min at 72° C. for 35 cycles, followed by 8 min at 72° C. PCR products were analyzed on 2% agarose gel containing ethidium bromide. Following electrophoresis, PCR products were transferred to Hybond N+ membrane (Amersham, Arlington Heights, Ill.) in 20×SSC overnight and immobilized using 0.4 M NaOH. Blots were hybridized with ³²P-γATP labeled oligonucleotide probes in Rapid—hyb buffer (Amersham) for 1 h at 42° C. Each cytokine primer mix was used as a radiolabeled probe (as per instructions). Equivalent loading was assessed following hybridization with a probe generated from pHE7 sense primer. Washed blots were then analyzed using PhosphorImager.

Effect of Ribavirin on Extracellular Cytokine Levels in Activated T Cells

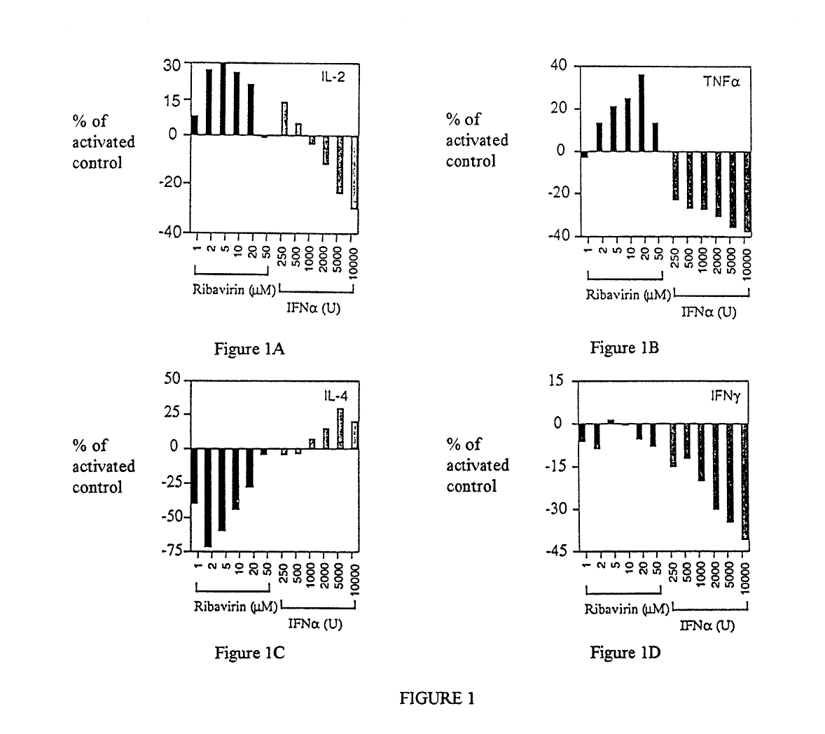

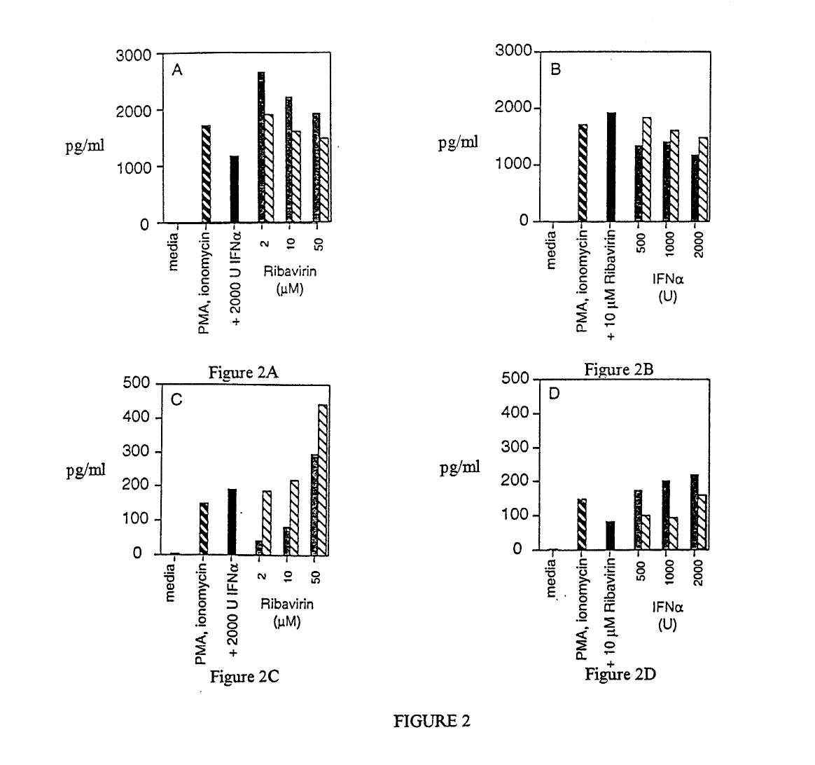

PMA/ionomycin treatment (48-72h) of human T-cells substantially increased the levels of all the cytokines analyzed i.e. IL-2, IL-4, TNFα, IFNγ (Table 1). The first number in each cell depicts the arithmetic mean, and the numbers in parenthesis depicts the relevant ranges. N=4. In a representative experiment shown in FIG. 1, addition of Ribavirin, in the dose range 0.5-50 μM, augmented activated levels of the Type 1 cytokines, IL-2 and TNFα maximally at 5 μM (30%) and 20 μM (36%) respectively. In contrast, interferon-α, inhibited IL-2 and TNFα expression in a dose-dependent manner (range 250-10000 U/ml, maximal inhibition 33 and 38% respectively), when compared to levels in untreated activated T cells. In addition, Ribavirin mediated a concomitant decrease in activated levels of the Type 2 cytokine, IL-4 (peak inhibition of 74% at 2 μM) whereas interferon-a maximally increased extracellular IL-4 by 26% (10000 U/ml). Using combinations of Ribavirin and interferon alpha, FIG. 2 shows that a constant 2000 U/ml of interferon alpha suppressed the Ribavirin dose-dependent augmentation of activated IL-2 levels (A) and reversed the inhibition of activated IL-4 levels (C). Similarly, a constant 10 μM of Ribavirin reversed the interferon alpha-mediated dose-dependent inhibition of activated IL-2 levels (B) and suppressed the augmentation of activated IL-4 levels (D).

Effect of Ribavirin on Cytokine mRNA Levels in Activated T Cells

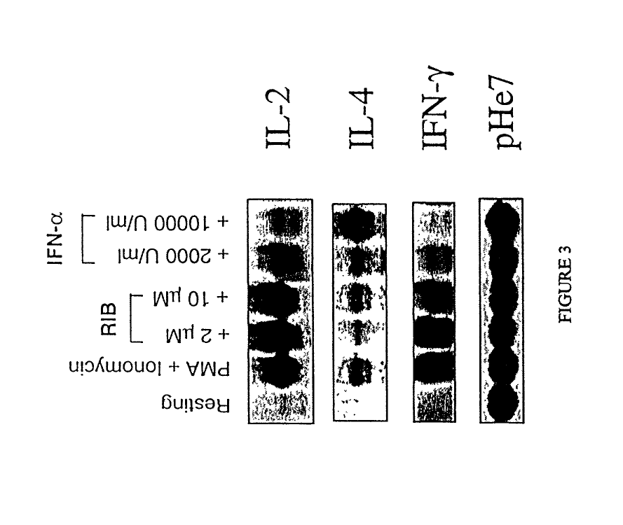

These opposing effects of Ribavirin and interferon-a on activated extracellular cytokine levels were also observed at the level of transcription. FIG. 3 shows that PMA/ionomycin treatment of human T-cells substantially augments IL-2, IL-4 and IFNγ MRNA levels. Treatment with Ribavirin (2, 5 and 10 μM) following T cell activation, elevates IL-2, decreases IL-4 and has no effect on IFNγ mRNA. In contrast, interferon α, at 1000, 2000 and 5000 U/ml decreases IL-2, increases IL-4 and decreases IFNγ mRNA. Therefore the respective dose-dependent effects of Ribavirin and interferon α on IL-2, TNFα, and IL-4 mRNA expression paralleled the ELISA analyses. These data suggest that Ribavirin promotes the synthesis of the Type 1 cytokines, IL-2 and TNFα and inhibits the expression of the Type 2 cytokine, IL-4 in activated human T cells.

Effect of Ribavirin on IL-2 and IL-4 Receptor Levels in Activated T Cells

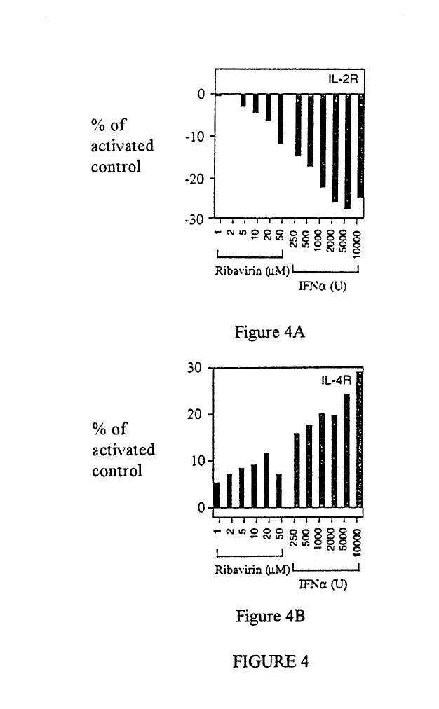

Using FACS analysis, we compared the effects of Ribavirin and interferon α on expression of IL-2 (CD2S) and IL-4 (CDw124) receptor expression in activated T cells. PMA/ionomycin-treatment increases CD25 and CDw124 expression from resting levels of 50.16±0.45 and 62.31±1.46 to activated levels of 162.48±2.89 and 87.53±3.98 respectively (n=4). In a representative of 3 experiments, FIG. 4 shows that Ribavirin (1-50 μM) has little effect on activated levels of L-2 and IL-4 receptor whereas interferon α in the dose range 250-10000 U/ml, decreased IL-2 receptor and increased IL-4 receptor expression in a dose-dependent manner, when compared to receptor levels in control activated T cells. Therefore, these data show that the effect of Ribavirin on cytokine synthesis acts independently of cytokine receptor expression. In contrast, the effect of interferon a treatment on IL-2 and IL-4 receptor correlates with that observed with its effect on activated IL-2 and IL-4 expression.

Effect of Ribavirin on Intracellular IL-2 Levels in CD4 and CD8⁺ Subsets of Activated T Cells

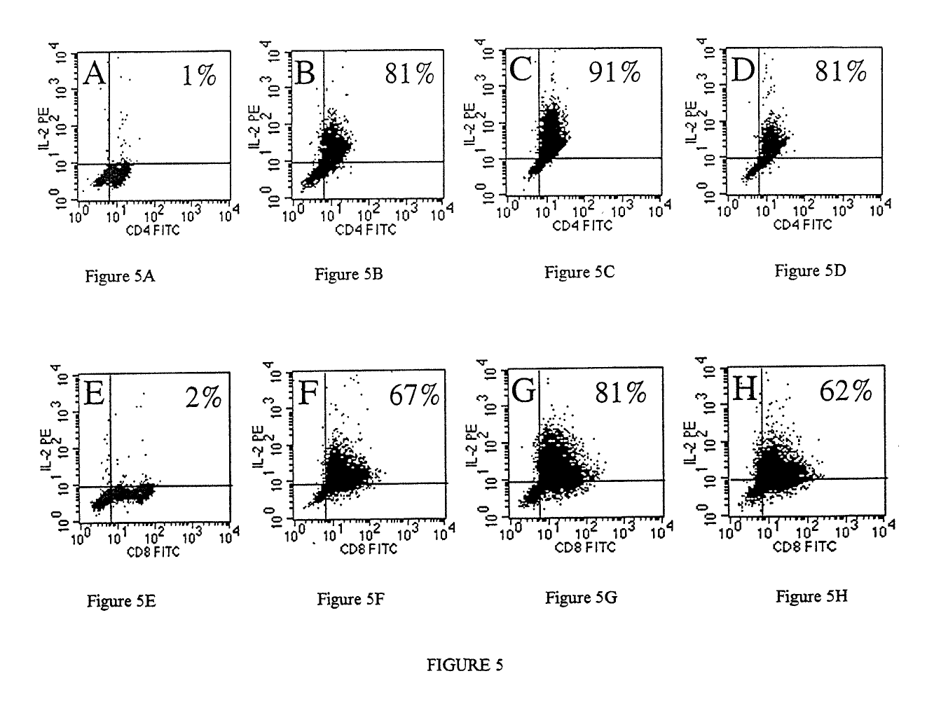

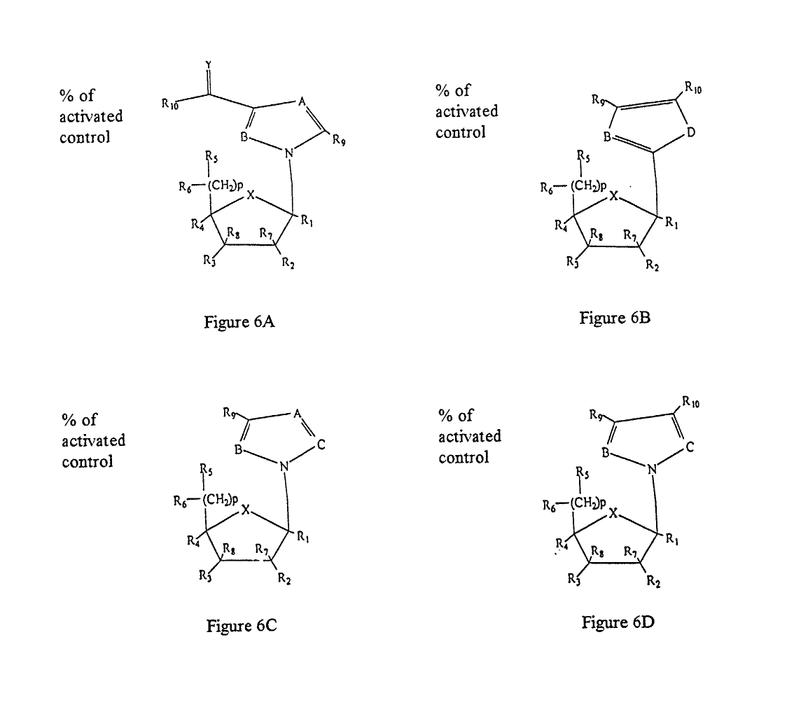

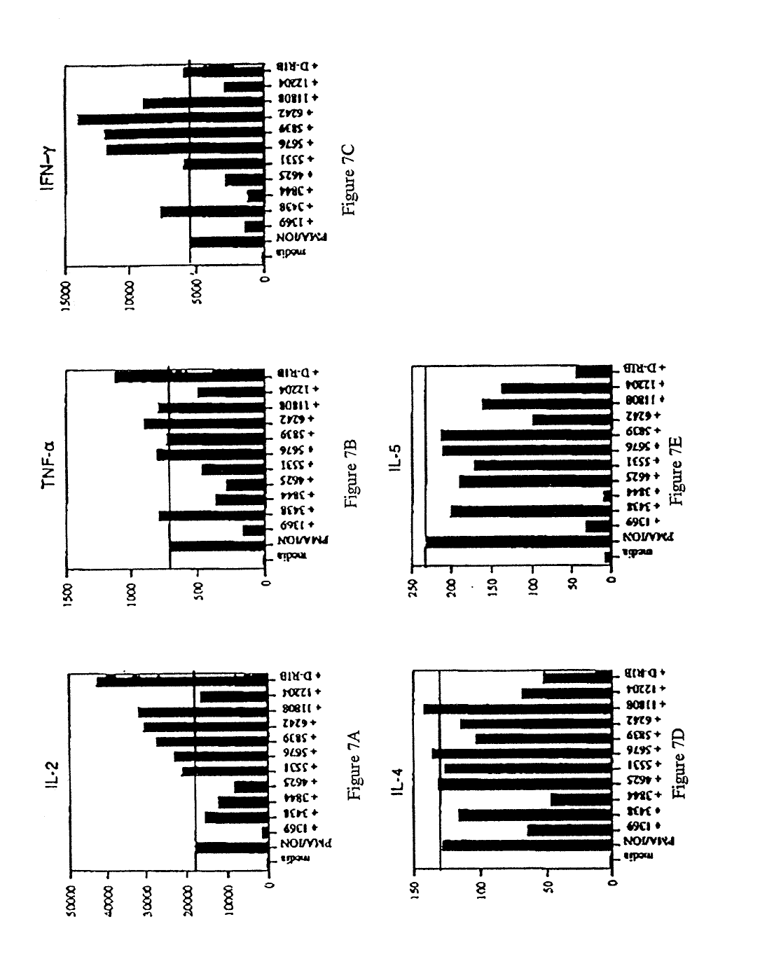

We examined whether the effect of Ribavirin on IL-2 expression was specific to CD4⁺ or CD8⁺ T cells. Intracellular IL-2 expression in fixed and Permeabilized activated T cells was determined by two-color flow cytometry using fluorescence-labeled antibodies to CD4 or CD8 and to IL-2. FIG. 5 shows that following treatment with Ribavirin at 10 μM, the percentage of CD4⁺ T cells expressing IL-2 rose from 82 to 91% and the percentage of CD8⁺expressing IL-2 increased from 81 to 91%. In contrast, the percentage of IL-2-expressing CD4⁺ and CD8⁺ cells following interferon a treatment (5000 U/ml) was 81 and 71% respectively. These data suggest Ribavirin has an effect on intracellular IL-2 expression which does not discriminate between CD4⁺ or CD8⁺ T cell subsets. In contrast, interferon a treatment has little effect on CD4⁺ T cells and even reduces IL-2 expression in the CD8⁺ T cell subset.

Thus, methods have been disclosed which employ nucleosides and other compounds to selectively modulate Type 1 and Type 2 responses relative to each other, especially in the treatment of disease. While specific embodiments have been disclosed herein, the scope of the invention is not to be limited except through interpretation of the appended claims.

What is claimed is:

1. A method of modulating Type 1 and Type 2 response in activated T cells of a human patient comprising administering ribavirin to the T cells in a dosage which promotes the Type 1 response and suppresses the Type 2 response.

2. The method of claim 1 wherein the amount of ribavirin added provides a concentration of about 0.25-6.7 μg/ml in a medium supporting the lymphocytes.

3. A method of treating a patient having a disease which includes a viral component and a non-viral component, the non-viral component being characterized by reduced Type 1 levels and increased Type 2 levels in activated T-lymphocytes, comprising administering ribavirin to the patient under a protocol sufficient to promote the Type 1 response and suppress the Type 2 response.

4. The method of claim 3 further comprising adding interferon alpha to the lymphocytes.

5. A method of inhibiting a virus by growing a virus in an environment having lymphocytes which produce Type 1 and Type 2 cytokine responses, and adding ribavirin to the environment in a concentration which increases the Type 1 response and suppresses the Type 2 response.

6. The method of claim 5 wherein the virus comprises Hepatitis C.
